(19) United States
(12) Patent Application Publication
(10) Pub. No.: US 2022/0297115 A1
(43) Pub. Date: Sep. 22, 2022

(54) FLUID PREPARATION DEVICES

(71) Applicant: Hewlett-Packard Development Company, L.P., Spring, TX (US)

(72) Inventors: Si-Lam J. CHOY, Corvallis, OR (US); Hilary ELY, Corvallis, OR (US)

(73) Assignee: Hewlett-Packard Development Company, L.P., Spring, TX (US)

(21) Appl. No.: 17/642,279

(22) PCT Filed: May 8, 2020
(86) PCT No.: PCT/US2020/032202

§ 371 Date: Mar. 11, 2022

Related U.S. Application Data
(63) Continuation of application No. PCT/US2019/058429, filed on Oct. 29, 2019.
Application No. PCT/US2019/058429 is a continuation of application No. PCT/US2019/058427, filed on Oct. 29, 2019.

(51) Int. Cl. B01L 3/00; C12N 15/10

(52) U.S. Cl. CPC B01L 3/502; C12N 15/1013; B01L 2200/0621; B01L 2200/0631; B01L 2200/0689; B01L 2300/087; B01L 2400/0406; B01L 2400/043

(57) ABSTRACT

A fluid preparation device can include a fluid-receiving vessel and a fluid loader with multiple fluid chambers including a first fluid chamber and a second fluid chamber. The multiple fluid chambers can partially be defined by actuator seals that are positioned on an actuator. The actuator can be moveable among a plurality of positions and the actuator seals can be positioned on the actuator to fluidically separate the first fluid chamber from the second fluid chamber when the actuator is in a closed position from the plurality of positions, allow fluidic communication between the second fluid chamber and the fluid-receiving vessel when the actuator is in a first open position from the plurality of positions, and allow fluidic communication between the first fluid chamber and the second fluid chamber when the actuator is in a second open position from the plurality of positions.

### BACKGROUND

In biomedical, chemical, and environmental testing, isolating a component of interest from a sample fluid can be useful. Such separations can permit analysis or amplification of a component of interest. As the quantity of available assays for components increases, so does the demand for the ability to isolate components of interest from sample fluids.

### BRIEF DESCRIPTION OF THE DRAWINGS

FIG. 1 illustrates an example fluid preparation device loaded with a particulate substrate and loadable with fluids shown at various stages of use in accordance with the present disclosure;

FIGS. 2A and 2B illustrate an example fluid preparation device with fluids shown at various stages of use in accordance with the present disclosure;

FIG. 3 illustrates an example fluid preparation device loaded or loadable with fluids in accordance with the present disclosure;

FIG. 4 illustrates an example fluid preparation device loaded or loadable with fluids which can be used to prepare a fluid column with both a multi-fluid density gradient and a capillary force-supported gradient in accordance with the present disclosure;

FIG. 5 illustrates an example series of fluid preparation device assembled as a manifold in accordance with the present disclosure; and

FIG. 6 is a flow diagram illustrating an example method of forming a multi-fluid density column in accordance with examples of the present disclosure.

### DETAILED DESCRIPTION

In biological assays, a biological component can be intermixed with other components in a biological sample that can interfere with subsequent analysis. As used herein, the term “biological component” can refer to materials of various types, including proteins, cells, cell nuclei, nucleic acids, bacteria, viruses, or the like, that can be present in a biological sample. A “biological sample” can refer to a fluid obtained for analysis from a living or deceased organism. Isolating the biological component from other components of the biological sample can permit subsequent analysis without interference and can increase accuracy of the subsequent analysis. In addition, isolating a biological component from other components in a biological sample can permit analysis of the biological component that would not be possible if the biological component remained in the biological sample. Many of the current isolation techniques can include repeatedly dispersing and re-aggregating samples. The repeated dispersing and re-aggregating can result in a loss of a quantity of the biological component. Furthermore, isolating a biological component with some of these techniques can be complex, time consuming, and labor intensive and can also result in less than maximum yields of the isolated biological component.

In accordance with examples of the present disclosure, a fluid preparation device includes a fluid-receiving vessel and a fluid loader with multiple fluid chambers including a first fluid chamber and a second fluid chamber. The multiple fluid chambers in this example are partially defined by actuator seals that are positioned on an actuator. The actuator moveable among a plurality of positions. The actuator seals are positioned on the actuator to fluidically separate the first fluid chamber from the second fluid chamber when the actuator is in a closed position from the plurality of positions, allow fluidic communication between the second fluid chamber and the fluid-receiving vessel when the actuator is in a first open position from the plurality of positions, and allow fluidic communication between the first fluid chamber and the second fluid chamber when the actuator is in a second open position from the plurality of positions. In one example, the first open position and the second open position can be the same position from the plurality of positions.

In further detail regarding the fluid preparation device, in another example, the first fluid chamber can be loaded with a first fluid and the second fluid chamber is loaded with a second fluid. In further detail, the first fluid chamber can loaded with a first fluid having a first fluid density, the second fluid chamber is loaded with a second fluid having a second fluid density that has a greater density than the first fluid density, and the first fluid or the second fluid includes a particulate substrate dispersed therein, wherein when the second fluid and the first fluid are received by the fluid-receiving vessel, a multi-fluid density gradient column is formed in the fluid-receiving vessel. The first fluid, for example, can include a biological sample including a biological component that can become associated with a surface of the particulate substrate. The particulate substrate can include magnetizing particle. In another example, fluid communication from the first fluid chamber to the second fluid chamber is established via a first bypass channel allowing drainage from the first channel around a first actuator seal, and fluid communication from the second fluid chamber to the fluid-receiving vessel is established via a second bypass channel allowing drainage from the second channel around a second actuator seal. The first fluid chamber, the second fluid chamber, and the fluid-receiving vessel can also include vents to permit air flow when the actuator is in the first open position or the second open position. In another example, a magnet can be included that is spatially located adjacent to the fluid-receiving vessel to provide a magnetic field across the fluid-receiving vessel. In another example, the fluid-receiving vessel includes a capillary fluid gradient portion. The capillary fluid gradient portion can be loaded with a fluid and is layered adjacent to a second fluid along a capillary force-supported interface.

In another example, a particulate substrate fluid density layering system includes a first fluid having a first fluid density, a second fluid having a second fluid density that is denser than the first fluid density, and a particulate substrate dispersed or dispersible in the first fluid or the second fluid. The system in this example also includes a fluid loader with multiple fluid chambers partially defined by actuator seals that are positioned on a common actuator. When the actuator is in a closed position, a first fluid chamber containing the first fluid and a second fluid chamber containing the second fluid are isolated from one another, and when the actuator is in an open position, one or both of the first fluid or the second fluid is allowed to drain from the first fluid chamber or the second fluid chamber, respectively. The particulate substrate in one example can include magnetizing particles that are dispersed or dispersible in the first fluid or the second fluid. The magnetizing particles can include a surface so that a biological component of a biological sample or can become associated with the surface of the magnetizing particles. In another example, the first fluid chamber can be pre-loaded with the first fluid which includes magnetizing particles dispersed therein, or the first fluid chamber can be pre-loaded with dry magnetizing particles and the first fluid is loadable into the first fluid chamber so that the first fluid includes the magnetizing particles dispersed therein. In one example, a fluid-receiving vessel can be integrated with the fluid to receive the multi-fluid density gradient column above a pre-loaded elution buffer or a master mix for nucleic acid process positioned.

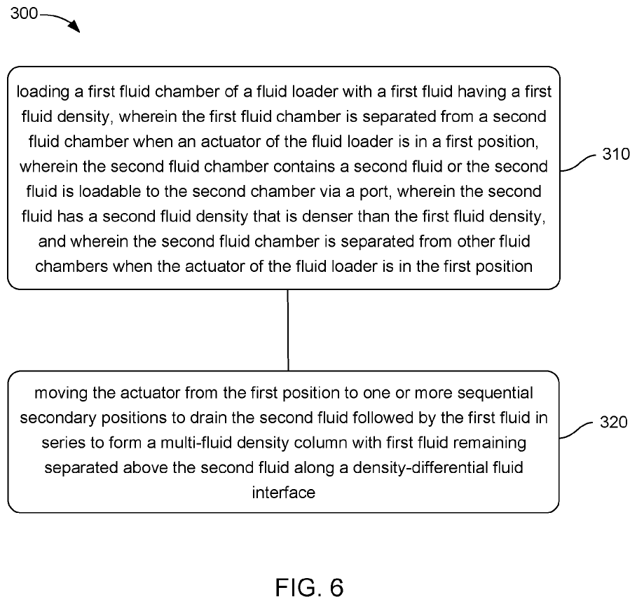

In another example, a method of forming a multi-fluid density column is shown in FIG. 6. The method in this example includes loading a first fluid chamber of a fluid loader with a first fluid having a first fluid density. The first fluid chamber is from a second fluid chamber when an actuator of the fluid loader is in a closed position. The second fluid chamber contains a second fluid or the second fluid is loadable to the second fluid chamber via a port, wherein the second fluid has a second fluid density that is denser than the first fluid density, and wherein the second fluid chamber is separated from other fluid chambers when the actuator of the fluid loader is in the closed. In further detail, the method includes moving the actuator from the closed position to one or more sequential open positions to drain the second fluid followed by the first fluid in series to form a multi-fluid density column with first fluid remaining separated above the second fluid along a density-differential fluid interface. In further detail, the method can include combining the first fluid with a particulate substrate either in the first fluid chamber or prior to loading the first fluid to the first fluid chamber, wherein the particulate substrate includes a surface to become associated with a biological component of a biological sample or is associated with the biological component of the biological sample. The multi-fluid density gradient column can be formed in a fluid-receiving vessel positioned adjacent to a magnet, wherein the magnet is actuatable to move magnetizing particles included in the first fluid or the second fluid along the multi-fluid density gradient column.

It is noted that when discussing examples of fluid preparation devices, particulate substrate fluid density layering systems, or methods of forming a multi-fluid density column, such discussions can be considered applicable to one another whether or not they are explicitly discussed in the context of that example. Thus, for example, when discussing a fluid preparation device, such disclosure is also relevant to and directly supported in the context of a particulate substrate fluid density layering system, or a method of forming a multi-fluid density column, and vice versa.

Terms used herein will have the ordinary meaning in the relevant technical field unless specified otherwise. In some instances, there are terms defined more specifically throughout the specification or included at the end of the present specification, and thus, these terms can have a meaning as described herein.

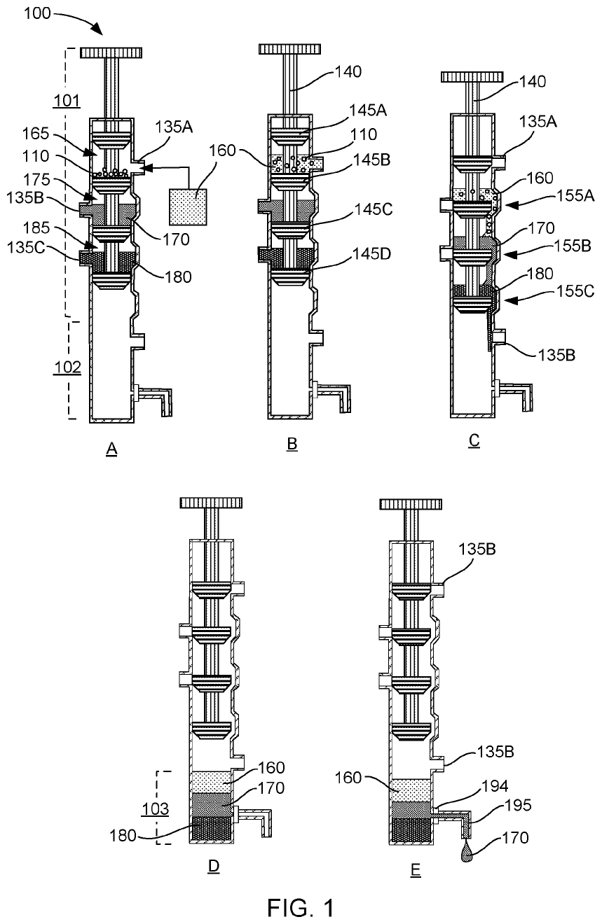

FIG. 1 illustrates a fluid preparation device 100 that can be used with a particulate substrate 110 and various fluids 160, 170, 180. The fluid preparation device can include a fluid loader 101 and a fluid-receiving vessel 102 for receiving fluids from the fluid loader to form a fluid mixture or a multi-fluid density gradient column therein. Thus, it is noted that the present disclosure describes the preparation of fluids that can either be mixed or that can be layered along a fluid gradient, for example. Thus, fluids can be combined in series to be mixed together where there is applicability of applying a first fluid in series over a second fluid, e.g., gentle mixing or for some other reason where combining fluids sequentially would be beneficial. However, in accordance with one example, the fluids may be likewise combined in series when there are applications suitable for forming fluid layers that remain phase separated, such as may be the case in a vertically layered fluid column. With that example in mind, the figures presented herein related for the most part to forming vertically layered fluid columns, e.g., multi-fluid density gradient columns or multi-fluid density gradient columns with other fluids that may be included in other forms, such as layered fluids with one or multiple fluids remaining phase separated in part due to capillary forces relative to the fluidic surface tension and the vessel cross-sectional size at the fluid interface, as will be described in some detail hereinafter. With that stated, it is understood that the present disclosure also relates to fluid mixing or other fluid combing applications that would benefit from the use of the fluid preparation devices of the present disclosure, even though the figures provide some focus on fluid layering.

In accordance with this, as shown in FIG. 1, an example loading sequence is shown where fluid preparation device 100 and related system is shown at various points based on various loading stages and actuator positions, notated as columns stages A to E. This example is not considered to be limiting, but shows the various examples of loading and fluid flow, e.g., draining, that occurs in series to form a fluid mixture or as shown in example, a multi-fluid density gradient column, in a fluid-receiving vessel 102 below a fluid loader 101. It is noted that the fluid-receiving vessel in this example is integrated with the fluid loader, but the fluid-receiving vessel could be separate or modularly joinable with the fluid loader, for example. As shown in FIG. 1 at column stage “A”, the example fluid preparation device 100 is shown as being loadable with a first fluid 160 having a first fluid density, containing a second fluid 170 having a second fluid density that is denser than the first fluid density, and in this specific example, containing a third fluid 180 having a third fluid density that is denser than the second fluid density. If the third fluid relies on capillary forces to retain its fluidic separation between the third fluid and the second fluid in the fluid-receiving column, then the third fluid may be less dense than the second fluid. However, in this example, the fluid-receiving vessel may not be configured narrowly enough at the bottom to benefit from establishing a capillary force-supported interface (as described in greater detail hereinafter). With that said, with an appropriately constructed or shaped fluid-receiving vessel including dimensional cross-sectional size relative to the surface tension of the fluids at the fluid interface, the third fluid could be an oil or some other light oil that is less dense than the second fluid if the fluid-receiving vessel is configured to provide capillary force support to maintain the fluid interface.

In further detail, the fluid loader 101 (which is a portion of the fluid preparation device) includes multiple fluid chambers 165, 175, 185 that are partially defined by actuator seals 145A, 145B, 145C, and 145D fixed along a common actuator 140 (labeled in column “B”). In this instance, the actuator and the actuator seals are part of a plunger device, and the fluid loader is configured similarly to a syringe barrel, but with several modifications. As configured, when the actuator, e.g., plunger shaft, is in a closed position, the first fluid chamber 165 to contain the first fluid 160 and the second fluid chamber 175 to contain the second fluid 170 are isolated from one another, as shown. This is also the case with the third fluid 180 contained within the third fluid chamber 185. In this particular example, the particulate substrate 110 (which can be magnetizing particles, for example), are shown as pre-loaded in the first fluid chamber and the first fluid is shown as being loadable in the first fluid chamber via loading port 135A to admix with and disperse the particulate substrate (shown at column “B”). Other loading ports are shown at 135B and 135C, such as for loading fluid chamber 175 and/or fluid chamber 185, but it is noted that these chambers can alternatively be pre-loaded with fluid.

In the example shown in FIG. 1, at column stage “C,” the actuator 140 is shown as being depressed into an open position. In this configuration, fluid 180, fluid 170, and fluid 160 can drain simultaneously to sequentially form the multi-fluid density gradient column 103 shown at column “D.” In further detail regarding column “C,” it is noted that the loading port 135A (shown at column “A”) is now positioned to act as a drainage vent. Thus, airflow is allowed into the first fluid chamber so that the draining fluid can be vented from both above and below via vent 135B found in the fluid-receiving vessel 102 region. In some examples, the vents can be pressurized (positively or negatively) to assist with fluid flow/drainage. Furthermore, in this example, the drainage is allowed by the formation of gaps between the actuator seals 145B, 145C, and 145D and a side wall of the fluid loader barrel, which in this example can be in the form of bypass channels 155A, 155B, 155C. Bypass channel 155A is associated with fluid 160 drainage, bypass channel 155B is associated with fluid 170 drainage followed by fluid 160 drainage, and bypass channel 155C is associated with fluid 180 drainage followed by fluid 170 drainage followed by fluid 160 drainage. Alternatively, the gap or bypass channel can be in the form of a side rib at a side wall that tents or lifts the seal from the side wall, creating the gap on either side of the rib, for example.

Referring now to column stage “E,” the multi-fluid density gradient formed can then be processed downstream as may be applicable. In this instance, there is a fluid evacuation port or nozzle 195 that is opened via valve 194 to remove one or more of the fluids. In this instance, the fluid evacuation port is shown as being suitable for removing the middle fluid (fluid 170) or sequentially both the middle fluid and the top fluid (fluid 160). The fluid evacuation port or nozzle can be at any location as may be useful or applicable for a given application.

In further detail regarding the actuator seals 145A, 145B, 145C, and 145D, they are positioned to fluidically separate the first fluid chamber from the second fluid chamber when the actuator is in a closed position from the plurality of positions. That is shown at column stage “B” for example. Furthermore, the actuators seals also allow for fluidic communication between the second fluid chamber and the fluid-receiving vessel when the actuator is in a first open position from the plurality of positions, and allow for fluidic communication between the first fluid chamber and the second fluid chamber when the actuator is in a second open position from the plurality of positions. That is show at column stage C in this example.

It is noted that the term “first,” “second,” “third,” etc., are used for clarity in describing certain figures and for understanding the disclosure but should not be considered to be limiting. For example, fluid 180 could be referred to as a “first fluid,” or a “second fluid” or a “third fluid.” This is also the case with other features where similar “first,” “second,” “third,” etc. naming conventions are used.

Furthermore, the terms “density gradient” or “multi-fluid density gradient” can be used in various contexts herein but refer to the ability of multiple fluids to remain separated in layers due to their density difference (with denser fluids being positioned vertically lower along the column). Thus, there can be multiple fluids that are phase separated, but are still in direct contact at a fluid interface, referred to herein as a “density-differential interface,” which is descriptive of the interface being present as a result of the density difference.

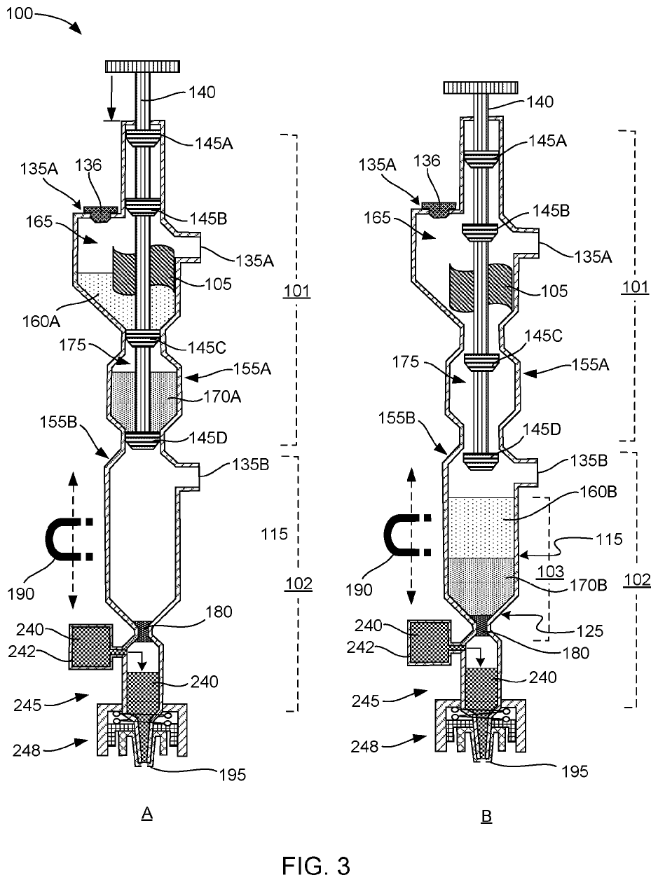

On the other hand, the terms “capillary force” or “capillary force-supported gradient” refer to fluid interfaces that are not provided by their increasing density and their density difference, but rather, the fluids of immediately adjacent layers can have different densities, but less dense fluids can be positioned below denser fluids, and the reason these less dense fluids do not migrate upward is because they are constrained within a narrow fluidic channel due to the surface tension interaction between the fluid at the fluid interface, namely at the “capillary force-supported interface.” In accordance with the present disclosure, the fluid columns described herein that can be used include multi-fluid density gradient columns or column portions, but in some examples, may also include capillary force gradient portions. FIGS. 1-3 show examples where a multi-fluid density gradient column is formed, whereas FIG. 4 shows an example where the column formed includes a multi-fluid density gradient portion as well as a capillary force gradient portion.

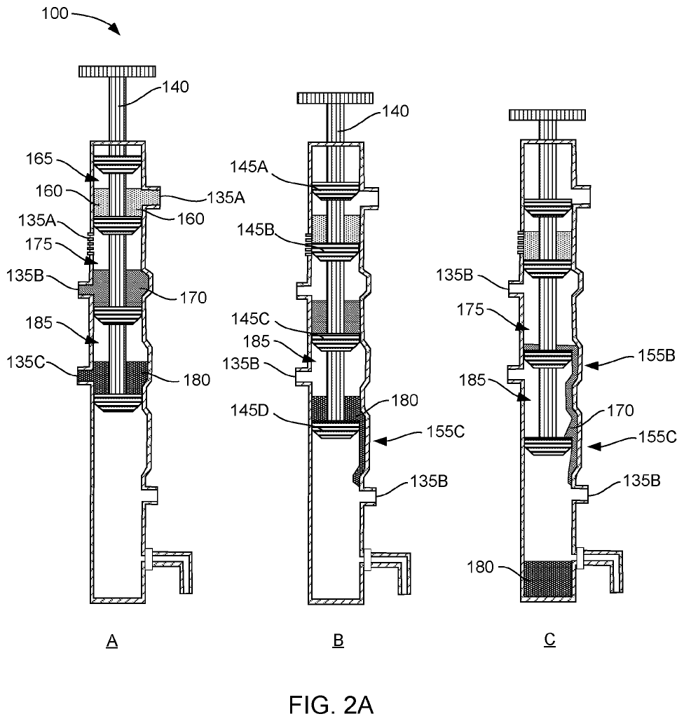

An alternative example fluid preparation device is shown at 100 in FIG. 2A and FIG. 2B with pre-loaded fluids. It is noted that the fluid preparation device does not include the fluids therein per se, but in some examples, can be pre-loaded with fluids or particulate substrates or the like. This example does not show the particulate substrate loaded or loadable in one or more of the fluids, but it is understood that the same details apply to this and other examples as if shown here as well. In this example, again various loading stages and actuator positions are shown at from A to E. For example, at column stages “A” and “B,” a first closed position is shown. At column stage “C,” at the actuator position shown in this example, a first open position and a second open position are simultaneously in place, as the second fluid 170 is open to the fluid-receiving vessel 102 (through the third fluid chamber 180) and the first fluid 160 is open to the second fluid chamber 175. This example is not considered to be limiting, but shows the various examples of loading and draining fluids sequentially to form a multi-fluid density gradient column in a fluid-receiving vessel below a fluid loader. More specifically, in FIG. 2A, at column stage “A,” the example fluid preparation device is shown as including a first fluid 160 having a first fluid density, a second fluid 170 having a second fluid density that is denser than the first fluid density, and in this specific example, a third fluid having a third fluid density that is denser than the second fluid density. The fluid loader (which is a portion of the fluid preparation device) includes multiple fluid chambers 165, 175, 185 that are partially defined by actuator seals 145A, 145B, 145C, and 145D which are fixed along a common actuator 140. In this instance, the actuator and the actuator seals are part of a plunger device, and the fluid loader is configured similarly to a syringe barrel, but with several modifications. As configured, when the actuator, e.g., plunger shaft, is in a closed position, the first fluid chamber 165 to contain the first fluid 160 and the second fluid chamber 175 to contain the second fluid 170 are isolated from one another, as shown. This is also the case with the third fluid 180 contained within the third fluid chamber 185. In this particular example, the fluids are shown as pre-loaded in the respective chambers, but could be loadable into the device through ports 135A, 135B, and 135C.

In the example shown in FIG. 2A, at column stage “B,” the actuator 140 is shown as being depressed into a first open position and a second open position is shown by example at column “C.” It is noted that in this particular embodiment, the second open may alternatively be an intermediate position that occurs between the first open position and a third open position. That is because the second position described herein occurs when one of the fluid channels is open to another of the fluid channels, regardless of whether they are referred to herein as “first,” “second,” “third,” etc. Thus, the third fluid can drain to the fluid-receiving vessel in the first open position and the second fluid can drain to the third fluid chamber in the second open position. Alternatively, the second fluid can be allowed to drain all the way to the fluid-receiving vessel in the first open position (through the third fluid chamber) and the first fluid can be allowed to drain into the second fluid chamber in the second open position. Stated another way, passageways have been opened to allow multiple fluids to flow through the passageways at various actuator positions. In accordance with this, the terms “first,” “second,” “third,” etc. are relative terms and any two fluids or actuator positions could be referred to “first” and “second” relative to one another. With that in mind, in the configuration shown at column “B,” fluid 180 can drain around actuator seal 145D via bypass channel 155C to form a first lowermost layer of the multi-fluid density gradient column, shown at column “C.” Fluid 170 and fluid 180 remain in their respective fluid chambers, as the bypass channels associated with their respective fluid chambers have not been opened with the actuator at the third position.

In further detail regarding the actuator seals 145A, 145B, 145C, and 145D, it is noted that the as the actuator 140 is moved to different positions, the seals in this example also move along with the actuator, and thus, the movement of the seals acts to open and close the chambers that the specific seals define (either to drainage below, or in some instances, to venting from above if a vent is not included in the chamber at the position where the fluid may be allowed to drain). In this example, when bypass channels 155A, 155B, and 155C are closed, the seals are fitted against walls of the fluid loader, and when the bypass channels are opened, there is separation between the seals and the walls of the fluid loader.

In further detail regarding column “C,” when the actuator is in a “second” open position, fluid 170 is allowed to drain through bypass channel 155B and then through 155C to form a second layer of the multi-fluid density gradient column, shown at column “D” in FIG. 2C. Also shown at column “D,” the actuator is then depressed to an alternative “second” open position. In this position, the first fluid 160 is allowed to pass or drain through bypass channel 155A, and then through bypass channel 155B, and then followed by drainage channel 155C to form a third layer of the multi-fluid density gradient column (shown at column “E”). It is noted that venting is provided for drainage at the various vents 135A shown, depending on when the respective fluids are available for draining about the bypass channels, as described above. In further detail, with respect to column stage “E,” the multi-fluid density gradient column formed can then be processed downstream as may be applicable. In this instance, there is a fluid evacuation port or nozzle 195 that can be fluidly coupled to a valve 194 to allow for removal of one or more of the fluids. In this instance, the fluid evacuation port is shown as being suitable for removing the middle fluid (fluid 170) or sequentially both the middle fluid and the top fluid (fluid 160). The fluid evacuation port or nozzle can be at any location as may be useful or applicable for a given application.

In further detail regarding the actuator seals 145A, 145B, 145C, and 145D, they are positioned to fluidically separate the first fluid chamber from the second fluid chamber when the actuator is in a closed position from the plurality of positions. Thus, the closed position is shown at column stage “A” for example. Furthermore, the actuators seals also allow for fluidic communication between the second fluid chamber and the fluid-receiving vessel when the actuator is in a first open position from the plurality of positions. That is shown by way of example at column stage B. The actuator seals also allow for fluidic communication between the first fluid chamber and the second fluid chamber when the actuator is in a second open position from the plurality of positions. That is show at column stage “C” in this example.

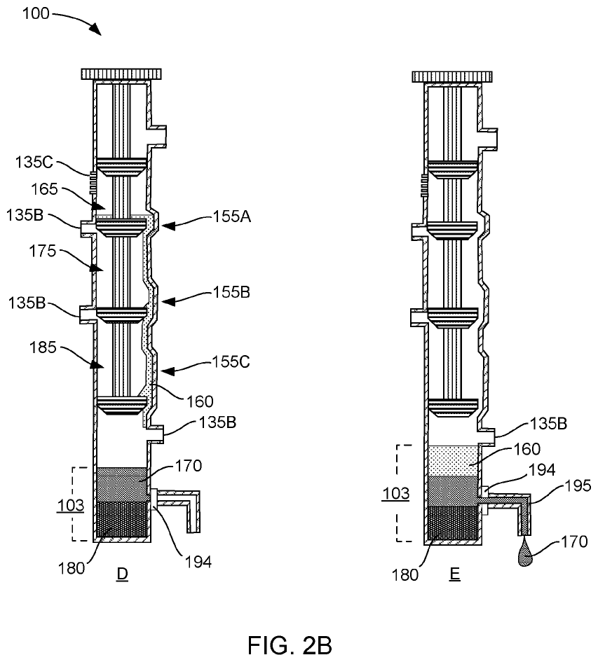

Turning now to FIG. 3, an alternative example of a fluid preparation device 100 and related system is shown that has a different construction with additional ports, features, geometries, etc., than that described previously. This example shows the fluid preparation device at two stages with the actuator 140 at two different positions, notated as column stages A and B, where the actuator is in a first closed position at column stage “A,” and in a first open position at column stage “B.” For example, fluid 160A is shown as being loaded in fluid chamber 165 at column stage A, and the same fluid is shown as fluid 160B at column stage B after it has been drained to generate a fluid layer along the multi-fluid density column with the fluid-receiving vessel (portion) 102 positioned beneath the fluid loader 101. Likewise, fluid 170A and fluid 170B are shown in two locations, but represent the same fluid at two different stages. In further detail, fluid 160A, 160B has a first fluid density, and fluid 170A, 170B has a second fluid density that has a greater density than the first fluid density. The fluid loader (which is a portion of the fluid preparation device that initially contains fluids 160A and 170A) includes multiple fluid chambers 165, 175 that are partially defined by actuator seals 145A, 145B, 145C, and 145D fixed along a common actuator 140. In this instance, the actuator and the actuator seals are part of a plunger device, and the fluid loader is configured similarly to a syringe barrel, but with some modifications. As configured, when the actuator, e.g., plunger shaft, is in a first closed position, the first fluid chamber 165 to contain the first fluid 160A and the second fluid chamber 175 to contain the second fluid 170A are isolated from one another, as shown. A particulate substrate (not shown in this figure, but shown in FIG. 1) can be pre-loaded in the first fluid chamber or can be loadable in the first fluid chamber and mixed as part of the first fluid either prior to or when in the first fluid chamber. Loading can occur through loading port 135A associated with the first fluid chamber. This loading port is shown as including a port cap 136 that may be removable and replaceable, or may allow for fluids to be injected there through, for example. Also shown in chamber 165 are a pair of rotary mixing paddles 105, which may be used by rotating the actuator to mix fluid 160 with the particulate substrate within the chamber, for example. There is also a vent 135A present to allow for fluid 160A to fluidly drain from the first fluid chamber when the plunger is depressed from a first closed position to a first open position.

As with prior examples, when the actuator 140 (or plunger in this example) is depressed from a closed position to a first open position, fluid 170A and fluid 160A can drain in series to form the multi-fluid density gradient column 103 in the fluid-receiving vessel 102 that includes the fluids at a new location, shown 170B and 160B. While draining, vents 135A above and below fluids 160A and 170A can provide air flow to allow for fluid flow around the actuator seals. In this instance, bypass channel 155A is provided by an enlarged barrel body rather than by a single channel, and bypass channel 155B is provided by an enlarged barrel body of the fluid-receiving vessel that receives the fluids from the fluid loader 101.

In the formation of the multi-fluid density gradient column 103 in the fluid-receiving vessel 102, the various fluids and/or particulate substrate carried by one or more of the fluids can be manipulated in various ways. In one example, if the particulate substrate can include magnetizing particles (such as magnetizing particles with a biological component associated with, e.g., attached, bonded, etc., to a surface thereof), the magnet 190 can be used to draw the particles through any of a number of density-differential fluid interfaces 115, for example (As a note, this this figure illustrates density-differential interfaces between fluids 160A and 170A, but also may include a capillary force-supported interfaces 125 if the cross-sectional area at the interface is sufficiently constrained to promoted fluids 170B and 180 staying separated, even if fluid 180 has lower density than fluid 170B. Furthermore, in addition to fluid 180, there may be a fluid or fluids that are pre-loaded or loadable in the fluid-receiving vessel, such as fluid 240. Fluid 240 in particular is shown as either being pre-loaded in a dispensing tip 245 of the fluid-receiving vessel or loadable from a fluidly connected loading apparatus 242. For example, the loading apparatus may be a container where the fluid is injected therein, or may have another configuration such as a blister pack, another syringe/plunger system, or the like, for example. To illustrate, a blister pack may include a fluid reagent that can be depressed to add to the column, for example. In the example shown, the dispensing tip can include a fluid evacuation port 195, which in this example is shown as being associated with a spring-loaded connector 248, but alternatively, could be a simple dispensing tip that does not include the more complicated spring-loaded connector. The dispenser tip could be used, for example, to dispense one or more fluids from the fluid-receiving vessel for downstream processing. Fluid processing may include, for example, receiving fluids for processing using fluid pumps, such as an injection pump, a syringe pump, a diaphragm pump, a peristaltic pump, etc. Example fluid processing that may be carried out can include evaluating fluids using a sensor, such as photo sensor, a thermal sensor, an optical sensor, a fluid flow sensor, a chemical sensor, an electrochemical sensor, a MEMS, or a combination thereof.

Referring now to FIG. 4, the same details regarding the fluid loader 101 and the fluid-receiving vessel 102 are as previously described. As with the other examples, a fluid mixture or a multi-fluid density gradient column can be formed in the fluid-receiving vessel. However, in the example shown here, the first fluid 160 (delivered originally from fluid chamber 165) and the second fluid 170 (delivered originally from fluid chamber 175) are separated by density along a density-differential interface 115, which means the second fluid is denser than the first fluid sufficiently to remain separated along this interface. However, fluid 180, which can be delivered from chamber 185, may be less dense than fluid 170 and even fluid 160 in some examples. However, the third fluid 180 may remain separate from the second fluid along a capillary force-supported interface 125, provided the surface tension of the fluid at the interface is constrained by a cross-sectional area within the fluid-receiving vessel sufficiently so that the interface remains intact and the third fluid does not migrate up into the second fluid. Thus, in this example, there is both a multi-fluid density gradient portion 103 of the column and a capillary force gradient portion 104 of the column. Notably, fluid 120 could be more or less dense than fluid 180, as either the greater density of fluid 120 assists in keeping the fluids separated and/or the constrained area at that interface (between fluids 180 and 120) is held intact by capillary forces as well. In one specific example, fluid 180 may be an oil, such as mineral oil, and fluid 120 may be a gas, such as air. The gas would not be introduced from one of the fluid chambers, but rather would likely be introduced from below through a port (not shown), for example. Thus, any particulate substrates with biological components associated therewith could pass through the oil and into a gaseous chamber, such as an air gap chamber to provide for additional contaminant clearing prior to the particles being deposited into fluid 130, which may be an elution buffer, a master mix fluid, or some other fluid that can be used to process the biological component that may be introduced therein.

In this and prior examples that may include a multi-fluid density gradient column (or a portion of the column includes a multi-fluid density gradient), the differences of the first fluid relative to the second fluid (or any two fluids along the multi-fluid density gradient portion) can be from 50 mg/mL to 3 g/mL, from 100 mg/mL to 3 g/mL, from 500 mg/mL to 3 g/mL or from 1 g/mL to 3 g/mL, as is the case with the other similar density gradient columns or column portions. However, the multi-fluid density gradient column, which can alternatively be referred to as a vertically layered fluid column, may also include the third fluid 180 mentioned above, which can be less dense than the second fluid 170. Thus, in a more standard sized column, the third fluid would typically otherwise migrate up into or through the second fluid, destroying the interface between the second and third fluids. However, in the example shown, this is not the case. The third fluid is constrained by the cross-sectional size of the column structure (along the plane where the second fluid interfaces with the third fluid). Thus, the surface tension of the third fluid combined with the size constraint of the column at this interface in combination provide capillary force-supported interface 125, which promotes the second fluid and the third fluid remaining separated from one another. More specifically, the capillary force-supported interface can be contained within a fluidic channel having a cross-sectional dimension that is aligned with the capillary force-supported interface, and can range from 0.5 μm to 2 mm. This dimension can be a diameter dimension, or for non-circular geometries, this dimension can be the average cross-sectional dimension.

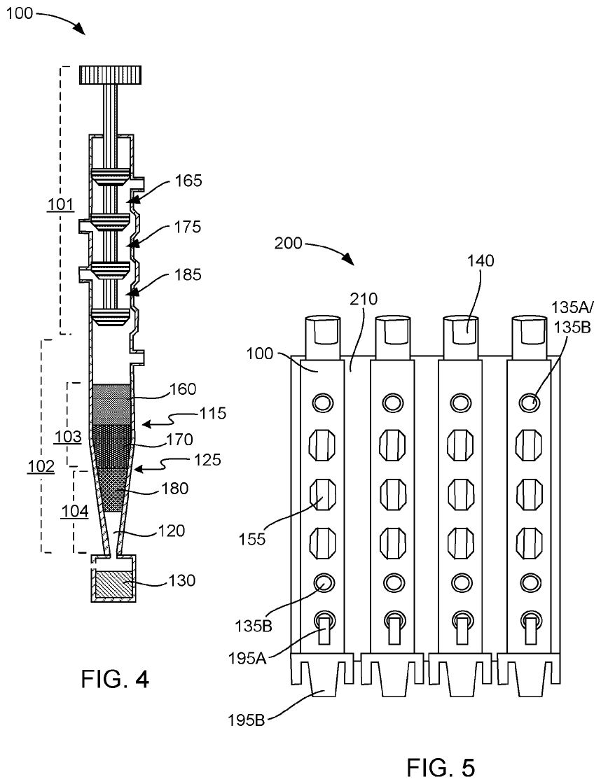

In further detail, as shown in FIG. 5, the fluid preparation device 100 can be part of a larger series of devices joined along manifold system 200. In this view, an outside of four devices is shown that are connected together along a support 210. Four is shown by way of example. The number of fluid preparation devices could range from 2 to 100, from 2 to 96, from 2 to 48, from 2 to 24, from 2 to 16, from 2 to 12, from 2 to 8, from 4 to 96, from 4 to 48 or from 4 to 6, for example. The actuators in this example are shown as having a head 142 that is engageable with an automated actuation system (not shown). A port for loading 135A which can also be used as a vent for facilitating fluid flow can be seen in this view, along with a plurality of bypass passageways 155. Another vent is also shown at 135B which is below the bypass passageways at the fluid-receiving vessel portion of the individual fluid preparation devices along the manifold. The two different fluid evacuation ports are shown at 195A (similar to the evacuation port shown in FIG. 1) and 1958 (similar to the evacuation port shown in FIG. 3), which is associated with a connector, such as a luer connector or some other mechanical connector.

With the examples shown in FIGS. 1-5, notably, these devices can be used with magnetizing particles. Thus, there are various strategies that can be used where one or multiple magnetic fields can be used to manipulate the location of the magnetizing particles along the fluid column formed (or fluid mixture formed if a mixture is formed). Movement can be in a positive or negative z-axis direction, but can also include movement along the x- and y-axes as well. Particle movement can be carried out, for example, using a magnet (which is inclusive of the use of multiple magnets). The magnet(s) can be, for example, permanent magnets and/or electrically induced magnetic elements, which can be at fixed positions or can be moveable about the column. Likewise, the column can be moved relative to the magnet(s). In another example, the magnet can be positioned adjacent to a side of the fluid-receiving vessel and can move vertically to cause the magnetizing particles to move therewith. In some examples, the magnet(s) can be moved along a side and/or along a bottom of the fluid-receiving vessel to pull the magnetizing particles in one direction or another. In one example, the magnet can be used to pull the magnetizing particles downward through fluid layers of a multi-fluid density gradient column or other fluids that may be present along the column. In yet other examples, the magnet can be used to concentrate the magnetizing particles near a side wall of the fluid-receiving vessel to be moved downward by a movable magnet, or by a magnet positioned beneath the fluid-receiving vessel. In one example, a magnet used to move magnetizing particles downward can be used to reverse the direction of the magnetizing particles and can cause the magnetizing particles to re-enter a fluid layer that the magnetizing particles have previously passed through.

A strength of the magnetic field and the location of the magnet in relation to the magnetizing particles can affect a rate at which the magnetizing particles move downward through the column. The further away the magnet and the lower the strength of the magnetic field, the slower the magnetizing particles will pass through the fluids along the column. In an example, a maximum distance between the magnet and a nearest location where one or more of the fluids resides along the fluid column can be about 50 mm maximum distance, about 40 mm maximum distance, about 30 mm maximum distance, about 20 mm maximum distance, or about 10 mm maximum distance. The minimum distance, on the other hand, may be from about 0.1 mm minimum distance, from about 1 mm minimum distance, or from about 5 mm minimum distance. In one example, the minimum distance between the magnet and the fluid column may be about the thickness of the container or fluid-receiving vessel that contains the fluid column. Thus, distance ranges between the magnet and the fluid column can be from about 0.1 mm to about 50 mm, from about 1 mm to about 50 mm, from about 1 mm to about 40 mm, from about 1 mm to about 30 mm, from about 1 mm to about 20 mm, from about 1 mm to about 10 mm, from about 5 mm to about 50 mm, or from about 5 mm to about 30 mm. In another example, a maximum distance between the magnet and a nearest location where one of the fluids resides along a multi-fluid density gradient portion of the column can be about 30 mm.

In further detail regarding the fluid-receiving vessels used, they can be configured as shown in the figures herein or can have other shapes. In one example, the fluid-receiving vessel can include a conical chamber and can also include a portion that provides capillary forces for a fluid to generate a capillary force-supported interface between fluids therein or between a fluid therein and a denser fluid positioned above. In one example, the capillary fluid gradient portion can be loaded with a fluid and is layered adjacent to a second fluid along a capillary force-supported interface. Thus, the fluid-receiving vessel can, in some examples, include capillary tube portion to provide vessel walls for the capillary force gradient portion. In further detail, the capillary tube portion can be fluidly coupled to a larger portion of the fluid-receiving vessel by a conical channel. The conical channel may have a cross-sectional size along the tapering channel that is narrow enough to support a capillary force-supported interface, for example. In further detail, shapes may include round cross-sectional tube shapes (for uniformly sized tubes as well as conical shaped portions). Notably, either or both could include a round, square, triangle, rectangle, or other polygonal cross-section with an appropriate capillary junction. Either or both could include bifurcations or other structures within the fluid-receiving vessel. The fluid-receiving vessel (and the fluid loader as well) may likewise include one or more input, output, or vent ports, and may or may not be symmetrical. Furthermore, the fluid-receiving vessel can be made of various polymers (e.g. Polypropylene, TYGON, PTFE, COC, others), glass (e.g. borosilicate), metal (e.g. stainless steel), or a combination of materials. Additionally, the capillary component could be formed from multiple materials used in various microfluidic devices, such as silicon, glass, SU-8, PDMS, a glass slide, a molded fluidic channel(s), 3-D printed material, and/or cut/etched or otherwise formed features. Film layers or other surface treatments can likewise be used for the structure as well. In further detail, the fluid-receiving vessel may be monolithic or a combination of components fitted together. The fluid-receiving vessel can be standalone or a component of a system (manual or automated) that includes functions or features for fluid positioning, particle manipulation, analysis, and/or other processes.

In further reference to the fluids used in the devices and systems described herein, they can have a density that is altered using a densifier. Example densifiers can include sucrose, polysaccharides such as FICOLL™ (commercially available from Millipore Sigma (USA)), C₁₉H₂₆I₃N₃O₉ such as NYCODENZ® (commercially available from Progen Biotechnik GmbH (Germany)) or HISTODENZ™, iodixanols such as OPTIPREP™ (both commercially available from Millipore Sigma (USA)), or combinations thereof. In one example, a density difference of the first fluid layer relative to the second fluid layer can range from about 50 mg/mL to about 3 g/mL. In yet other examples, a density difference from the first fluid layer relative to the second fluid layer can range from about 50 mg/mL to about 500 mg/mL or from about 250 mg/mL to about 1 g/mL. In further detail, example additives that can be included in the first fluid layer, or in other fluid layers, depending on the design of the multi-fluid gradient column, may include sucrose, C1-C4 alcohol, e.g., isopropyl alcohol, ethanol, etc., which can be included to adjust density, and/or to provide a function with respect to biological components to pass through the column.

A quantity of fluid layers along the fluid column is not particularly limited. The fluid columns can include, two fluids, three fluids, four fluids, five fluids, six fluids, seven fluids, etc.

Any of the fluids along the fluid column can be any of a number of combinations of fluids, such as gas fluid, e.g., air, aqueous fluid, non-polar fluid, polar, non-polar, miscible, or immiscible, etc. The fluids can be, for example a master mix fluid, reagent fluid, surfacing binding fluid, washing fluid, elution fluid, lysis fluid, etc. The fluids can likewise be pure, solutions, mixtures, suspensions, emulsions, and/or other forms. They may or may not undergo chemical reactions within the fluid-receiving vessel at any stage of the process, depending on the application. For example, a fluid in one layer can include a lysis buffer to lyse cells. In yet other examples, a fluid of another layer can be a surface binding fluid to bind the biological component to the magnetizing particles, a wash fluid to trap contaminants from a sample fluid and/or remove contaminants from an exterior surface of the magnetizing particles, a surfactant fluid to coat the magnetizing particles, a dye fluid, an elution fluid to remove the biological component from the magnetizing particles following extraction from the biological sample, a labeling fluid for binding labels to the biological component such as a fluorescent label (either attached to the magnetizing particles or unbound thereto), a reagent fluid to prep a biological component for further analysis such as a master mix fluid to prep a biological component for PCR, and so on.

In some examples, an individual fluid in one or multiple layers can provide sequential processing of a biological component from a biological sample. For example, individual fluids can carry out individual functions, and in many cases, the functions can be coordinated to achieve a specific result. Biological components that may be added can include whole blood, platelets, cells, lysed cells, cellular components, tissue, nucleic acids, e.g., DNA, RNA, primers, oligos, etc., or poly-bases, peptides, or the like. More specifically, for example, in considering biological components found in a cell, sequential fluid from top to bottom of a fluid column can act on the cell to lyse the cell in one of the fluids, and bind a target biological component from the lysed cell to magnetizing particles in a second fluid (or lysing and binding can alternatively be done in a single fluid). Additional fluid may be used to wash the magnetic microparticles with the biological component bound thereto in another fluid, e.g., washing the second fluid from magnetizing particles in the next fluid, and/or eluting (or separating) the biological component from the magnetizing particles in yet another lower layer. The surface binding and cell lysis can occur, for example, with a lysate buffer in a sucrose and water solution, e.g., the lysate (lysis) buffer can be densified with sucrose. Washing can occur in a sucrose in water solution, for example. In other examples, one or more of the fluids can be present as a fluid (layer(s)) along the fluid column in the form of a master mix fluid for nucleic acid processing. Other combinations of fluids (first, second, third, etc.) may include a surfacing binding fluid, a washing fluid, and an elution fluid; or may include a lysis fluid, a washing fluid, a surface binding fluid, a second washing fluid, an elution fluid, and a reagent fluid. Regardless of the various functions of the various fluids with sequentially increasing densities arranged from top to bottom, at the individual fluids, the magnetizing particles can independently interact, e.g., become modified, with fluids as layers in order to sequentially process the magnetizing particles with surface active groups and/or biological component associated therewith or associated with one or more of the fluids, for example.

A vertical height of the various layers of fluids along the fluid column can vary. Adjusting a vertical height of a fluid layer can affect a residence time of the paramagnetic microparticles in that fluid layer. The taller the fluid layer, the longer the residence time of the magnetizing particles in the fluid layer. In some examples, all of the fluid layers in the multi-fluid density gradient portion can be the same vertical height. In other examples, a vertical height of individual fluid layers can vary from one fluid layer to the next. In one example, a vertical height of the various layers along the fluid column can individually range from about 10 μm to about 50 mm. In another example, a vertical height of the fluid layers along the fluid column can individually range from about 10 μm to about 30 mm, from about 25 μm to about 1 mm, from about 200 μm to about 800 μm, or from about 1 mm to about 50 mm.

In further detail regarding the magnetizing particles (if used), these particles can include a surface that are associated with a biological component or can be formulated to become associated with a biological component in situ. Alternatively, the system can include a biological sample and the magnetizing particles (magnetizing particles or otherwise) can be surface-activated to preferentially bind with a biological component relative to secondary components in a biological fluid sample. Thus, the biological component may preferentially bind to the surface compared to secondary components such as enzymes, cellular debris, lysing agents, buffers, or a combination thereof. The magnetizing particles can be loaded in any of the fluid layers or preloaded with biological components bound to the surface of the magnetizing particles to be then dispersed in one or more of the fluids. Once in the multi-fluid density gradient column, the magnetizing particles can be moved vertically in either direction (up or down) using magnetic fields applied from both a particle aggregating profile and a particle sweeping profile. Movement in the x- and/or y-axes can also occur when aggregating and/or sweeping the magnetizing particles.

In further detail regarding the magnetizing particles, the magnetizing particles can be particles with a density suitable for gravity settling or centrifugation separation or movement along the column in a negative z-direction or may be buoyant to promote movement in a positive z-direction. Thus, when not acted upon with a magnetic field, they can still move within the fluids of the column in some instances.

The magnetizing particles can be in the form of paramagnetic microparticles, superparamagnetic microparticles, diamagnetic microparticles, or a combination thereof, for example. Whether using magnetizing particles or otherwise, the magnetizing particles can includes surfaces to become associated with a biological component, or can be associated with a biological component.

The term “associated” refers to any type of attach or adherence of a biological component with a surface of the particulate substrate. This can include covalent bonding, electrostatic or ionic attraction, surface adsorption, hydrogen bonding, and/or other adherence or linkage suitable for moving biological component together with the particulate substrate. In accordance with this, in some examples, the system can include a biological sample and the particulate substrate (magnetizing particles or otherwise) can be surface activated to preferentially bind with a biological component relative to secondary components in a biological fluid sample. Thus, the biological component may preferentially bind to the surface compared to secondary components such as enzymes, cellular debris, lysing agents, buffers, or a combination thereof. The particulate substrate can be loaded in any of the fluid layers and moved vertically in either direction (up or down).

In one example, the magnetizing particles can be surface-activated magnetizing particles that can be activated, for example, with surface groups that are interactive with a biological component of a biological sample or can include a covalently attached ligand attached to a surface of the magnetizing particles to likewise bind with a biological component of a biological sample. In some examples, the ligand can include proteins, antibodies, antigens, nucleic acid primers, amino groups, carboxyl groups, epoxy groups, tosyl groups, sulphydryl groups, or a combination thereof, or the like. Regarding combinations of ligands, there can be multiple types of ligands on common magnetizing particles, or a mixture of magnetizing particles with different ligands on multiple portions of the magnetizing particles (with the same or different magnetizing particles). The ligand can be selected to correspond with and bind with the biological component and can vary based on the type of biological component being isolated from the biological sample. For example, the ligand can include a nucleic acid primer when isolating a biological component that includes a nucleic acid sequence. In another example, the ligand can include an antibody when isolating a biological component that includes antigen. By way of example, commercially available examples of magnetizing particles with surface-activated groups include those sold under the trade name DYNABEADS®, available from ThermoFischer Scientific (USA).

In some examples, the magnetizing particles can have an average particle size that can range from about 0.1 μm to about 70 μm. The term “average particle size” describes a diameter or average diameter, which may vary, depending upon the morphology of the individual particle. The shape of the magnetizing particles can be spherical, irregular spherical, rounded, semi-rounded, discoidal, angular, sub-angular, cubic, cylindrical, or any combination thereof. In one example, the particles can include spherical particles, irregular spherical particles, or rounded particles. The shape of the magnetizing particles can be spherical and uniform, which can be defined herein as spherical or near-spherical, e.g., having a sphericity of >0.84. Thus, any individual particles having a sphericity of <0.84 are considered non-spherical (irregularly shaped). The particle size of the substantially spherical particle may be provided by its diameter, and the particle size of a non-spherical particle may be provided by its average diameter (e.g., the average of multiple dimensions across the particle) or by an effective diameter, e.g., the diameter of a sphere with the same mass and density as the non-spherical particle. In further examples, the average particle size of the magnetizing particles can range from about 1 μm to about 50 μm, from about 5 μm to about 25 μm, from about 0.1 μm to about 30 μm, from about 40 μm to about 60 μm, or from about 25 μm to about 50 μm.

In an example, the magnetizing particles can be unbound to a biological component when added directly to one of the fluid (layers) of a fluid column, in some instances initiating in one of the fluid chambers used to load the fluid receiving vessel. Binding between the magnetizing particles and the biological component of the biological sample can occur in one of the fluid chambers used for loading, or once present in the fluid column in the fluid-receiving vessel. In yet another example, the magnetizing particles and a biological sample including a biological component can be combined in a loading fluid before being added to one of the fluids at any point in time.

With more specific detail regarding the magnetizing particles, the term “magnetizing particles” is defined herein to include microparticles that may not be magnetic in nature unless and until a magnetic field is introduced at a strength and proximity to cause them to become magnetic. Their magnetic strength can be dependent on the magnetic field applied and may get stronger as the magnetic field is increased, or the magnetizing particles get closer to the magnetic source that is applying the magnetic field. In more specific detail, “paramagnetic microparticles” have these properties, in that they have the ability to increase in magnetism when a magnetic field is present; however, paramagnetic microparticles are not particularly magnetic when a magnetic field is not present. In some examples, the paramagnetic microparticles can exhibit no residual magnetism once the magnetic field is removed. The strength of magnetism of the paramagnetic microparticles can depend on the strength of the magnetic field, the distance between a source of the magnetic field and the paramagnetic microparticles, and a size of the paramagnetic microparticles. As a strength of the magnetic field increases and/or a size of the paramagnetic microparticles increases, the strength of the magnetism of the paramagnetic microparticles increases. As a distance between a source of the magnetic field and the paramagnetic microparticles increases, the strength of the magnetism of the paramagnetic microparticles decreases. “Superparamagnetic microparticles” can act similar to paramagnetic microparticles; however, they can exhibit magnetic susceptibility to a greater extent than paramagnetic microparticles in that the time it takes to become magnetized appears to be near zero seconds. “Diamagnetic microparticles,” on the other hand, can display magnetism due to a change in the orbital motion of electrons in the presence of a magnetic field.

### Methods of Forming Multi-fluid Density Columns

A flow diagram 300 of a method of forming a multi-fluid density column is shown in FIG. 6. The method can include loading 310 a first fluid chamber of a fluid loader with a first fluid having a first fluid density. The first fluid chamber can be separated from a second fluid chamber when an actuator of the fluid loader is in a closed position. The second fluid chamber can contain a second fluid or the second fluid is loadable to the second fluid chamber via a port, wherein the second fluid has a second fluid density that is denser than the first fluid density, and wherein the second fluid chamber is separated from other fluid chambers when the actuator of the fluid loader is in the closed. In further detail, the method can include moving 320 the actuator from the closed position to one or more sequential open positions to drain the second fluid followed by the first fluid in series to form a multi-fluid density column with first fluid remaining separated above the second fluid along a density-differential fluid interface. In further detail, the method can include combining the first fluid with a particulate substrate either in the first fluid chamber or prior to loading the first fluid to the first fluid chamber, wherein the particulate substrate is surface-activated to bind to a biological component of a biological sample or is surface-bound to a biological component of a biological sample. The multi-fluid density gradient column can be formed in a fluid-receiving vessel positioned adjacent to a magnet, wherein the magnet is actuatable to move magnetizing particles included in the first fluid or the second fluid along the multi-fluid density gradient column.

In some other examples, the biological sample including the biological component can be combined with the magnetizing particles in a loading solution prior to loading the biological sample including the biological component and the magnetizing particles into the multi-fluid density gradient portion. For example, the magnetizing particles and the biological sample can be mixed in a loading fluid. The biological sample and the magnetizing particles can be permitted to incubate or otherwise become prepared for loading on top of or into the multi-fluid density gradient column and/or other fluid along the fluid column. The magnetizing particles can bind with the biological component in the loading fluid and can then be added to the multi-fluid density gradient portion for the fluid layers to act upon the magnetizing particles. In one example, the loading fluid can become the uppermost fluid layer when loading from the top or can become the lowermost fluid layer when loading from the bottom, for example. Other fluid layers beneath or above the loading layer can be included through which the magnetizing particles are passed in part or in full.

The fluid used for loading the column (or the first fluid, or even the second fluid, or other fluid layer) can include secondary components selected from enzymes, cellular debris, lysing agents, buffers, or a combination thereof. The magnetizing particles can be bound to the biological component in a loading fluid or in a subsequent fluid along the multi-fluid density gradient portion. In the case of a loading fluid, magnetizing particles including the biological component bound thereto can then be introduced as a separate fluid layer for the microparticles to be drawn into other fluid layers that can act on the microfluidic particles to further interact with the surface thereof along the multi-fluid density gradient portion.

In accordance with the method, the magnetizing particles can be passed through multiple density-differential interfaces, and in some instances, a capillary force-supported interface, depending on the arrangement. The magnetizing particles can be passed through any or all of these interfaces from fluid to fluid in an upward or downward z-axis direction, though movement along the x- and y-axes also typically occurs during sweeping and aggregating.

In one example, the method can further include selectively withdrawing, e.g., pipetting, the biological component out of the third fluid layer, such as through an ingress/egress opening(s) from the top, the bottom, or through a sidewall, for example. The biological component may still be associated with a surface of the magnetizing particles or may be separated from the magnetizing particles. In another example, this method alternatively may include selectively withdrawing, e.g., pipetting, from one of the fluids (one of the layers), the second fluid layer, and/or the third fluid layer out of the multi-fluid density gradient portion and leaving the magnetizing particles with the biological component bound thereto in a fluid-receiving vessel of the multi-fluid density gradient portion to either be further treated or removed after the extraction of one of the fluids, the second fluid layer, and the third fluid layer therefrom. In some examples, the biological sample can include a cell with the biological component trapped within the cell (prior to lysis), a virus, or a biological component with extra-cellular vesicles. Lysing the cell can release the biological component therefrom and can permit isolation of the biological component. In this example, one of the fluids (or a loading fluid) can include a lysing agent for the cell. The method can further include lysing the cell in situ within one of the fluids or loading the fluid so that the biological component can be liberated from the cell and can bind with the magnetizing particles in one of the fluids (or fluid layers) or the loading fluid.

As another example, moving the magnetizing particles through the biological component separators can be carried out using any of a number of fluids in fluid layers, which can be layers of gas fluid, e.g., air, aqueous fluid, non-polar fluid, master mix fluid, reagent fluid, surfacing binding fluid, washing fluid, elution fluid, lysis fluid, etc. To illustrate, in a first fluid layer, lysing and particle binding may occur as cells, viral particles, or the like and are initially lysed and a nucleic acid is released into the first fluid. The nucleic acid can become bound to a surface of the magnetizing particles, which can be a magnetizing particle. Next, as the particles are drawn through a density-differential interface or a capillary force-supported interface and into a second fluid, cellular debris and unbound nucleic acid can be exchanged for a wash buffer fluid. A second wash can occur at the third fluid layer as bead-bound nucleic acid is further cleared of contaminants, e.g., lysed cellular debris and/or other contaminants or other material not of interest that may be present. Next, in some instances, oil exclusion may be carried out so that aqueous solution entrapped in the magnetizing particles can be replaced with oil, e.g., mineral oil. Other fluids may be suitable for this, but mineral oil is a good example of a fluid that may benefit from phase separation due to capillary forces in accordance with the present disclosure. Furthermore, by using an oil, this can provide an effective way of transitioning the magnetizing particles from being carried by a liquid fluid and passed into a gaseous fluid, such as at an air layer. Thus, the air layer or gap can be used to clear the contaminants further and can provide a mechanism to provide little to no contact between the fluids about the air gap. Magnetizing particles with entrapped mineral oil can be pulled into the air gap, providing reduced likelihood of concentration of contact between the wash and/or lysis buffer and the next liquid beneath the air gap, which can be, for example, an elution buffer, a master mix fluid for nucleic acid processing, or the like, for example. Other example processing sequences can likewise be used in accordance with the present disclosure.

To provide further example detail regarding how a vertically layered fluid columns prepared using the fluid preparation device described herein may be used process a biological component, for example, once a vertically layered fluid column is formed, such as that shown in FIG. 4, a biological material including cells, viral particles, or the like may be initially lysed and a nucleic acid is released in fluid 160, which may be a lysis-binding in fluid, for example. Lysis binding can be carried out using a solution including one or more of:
guanidine salt-based or other high salt content buffers used for solid phase extraction;
alcohol such as isopropyl alcohol (IPA), ethanol (EtOH), polyethylene glycol (PEG), or other suitable alcohols;
carrier nucleic acid(s);
enzymes to assist in lysis, such as Proteinase K, for example; and/or
pH adjuster to modify pH.

The nucleic acid from the cell can become bound to a surface of the magnetizing particles (not shown, but shown in FIG. 1 at 110). Thus, the magnetizing particles would be present in fluid 160 as shown in FIG. 4. Next, as the particles are drawn through interface 115, cellular debris and unbound nucleic acid can be exchanged for a wash buffer at fluid 170. A second wash can occur at fluid 180, as bead-bound nucleic acid is further cleared of contaminants, e.g., lysed cellular debris and/or other contaminants or other material not of interest that may be present. Example wash buffers for use at fluids 170 and/or 180 can be:
an aqueous solution including an alcohol, such as ethanol, and one or more of another alcohol, a binding agent binding agent, a salt, a surfactant, and/or a stabilizing agent; and/or
MyOne™ silane genomic DNA or viral kits, mRNA Direct kits, Mag Max™ kits, or other similar kits that include wash buffers often used with DYNABEADS®, all available from ThermoFischer Scientific, USA.

In further detail regarding, the capillary force gradient portion of the vertically layered fluid column, in this example, there are one or multiple fluids that can work together to clear debris from a particulate substrate as it is moved from the multi-fluid density gradient column portion (after the two layers of wash buffer) and into the capillary force gradient portion of the column. Those two fluids include the use of an oil and/or a gas (which can be present at fluid 120). Only one layer is shown, but there could be multiple layers. If an oil is used, the oil can be, for example, mineral oil. The gas can be, for example, air. Thus, the oil can be used for oil exclusion, where aqueous solution that may be present on or even entrapped in the particulate substrate (or magnetizing particles) can be replaced with the oil. Other fluids may be suitable for this, but mineral oil is a good example of a fluid that may benefit from separation due to capillary forces in accordance with the present disclosure. Furthermore, by using an oil, this can provide an effective way of transitioning the particulate substrate from being carried by a liquid fluid and passed into a gaseous fluid, such as air. Regarding the oil layer, specific oils that can be used include:
light oils, such as mineral oil for molecular biology or molecular grade mineral oils, light oil M5904 (density 0.84 g/mL at 25° C.) from Sigma-Aldrich (USA);
olive oil, such as high purity olive oil; and/or
densified oil.

Once the particulate substrate passes through the oil (fluid 120), if a gas is present as another layer there beneath, gas or air can be used to clear the contaminants further and can provide a mechanism to provide little to no contact between the fluids above and below the air gap. The biological component being separated (or further processed) has now have been loaded on the particulate substrate after cell lysis, washed in two different wash buffer layers, further contaminant-cleared by the oil, and passed through the air gap, providing reduced likelihood of concentration of contact between wash and/or lysis buffer and the next fluid beneath the air gap, e.g., elution buffer, a master mix fluid for nucleic acid processing, or the like. To separate the biological component, e.g., nucleic acid, from the particulate substrate, an elution buffer, shown by example at 130, can be used. Example elution buffers suitable for use may include one or more of:
aqueous salt solution (sufficient for elution but to retain biological component intact);
stabilizers;
surfactants; and/or
master mix if it is for a direct elution process, provide column is tuned for a target biological component of interest.

### Definitions

It is noted that, as used in this specification and the appended claims, the singular forms “a,” “an,” and “the” include plural referents unless the context clearly dictates otherwise.

As used herein, the term “about” is used to provide flexibility to a numerical range endpoint by providing that a given value may be “a little above” or “a little below” the endpoint. The degree of flexibility of this term can be dictated by the particular variable and determined based on experience and the associated description herein.

As used herein, the phrase “in direct contact” indicates that two or more fluids are fluidly coupled to one another, either directly or in some instances with intervening fluid(s) there between. In accordance with this definition, the term “in direct contact” excludes fluids that are separated by a physical barrier, but rather are phase separated by density or by using capillary forces as described herein, for example.

As used herein, the term “interact” or “interaction” as it relates to a surface of the magnetizing particles indicates that a chemical, physical, or electrical interaction occurs where magnetizing particles surface property is modified in some manner that is different than may have been present prior to entering the fluid layer, but does not include modification of magnetic properties or magnetizing particles as they are influenced by the magnetic field introduced by the magnet. For example, a fluid layer can include a lysis buffer to lyse cells, and cellular components can become associated with a surface of the magnetizing particles. Lysing cells in a fluid can modify the fluid sample and thus modify or interact with a surface of the magnetizing particles, e.g., the cellular component binds or becomes associated with a surface of the magnetizing particles. In yet other examples, a fluid layer that would be considered to interact with the magnetizing particles could be a wash fluid layer to trap contaminates from a sample fluid and/or remove contaminates from an exterior surface of the magnetizing particles, a surfactant fluid layer to coat the magnetizing particles, a dye fluid layer to introduce visible or other markers to the fluid or surface, an elution fluid layer to remove the biological component from the magnetizing particles following extraction from the biological sample, a labeling fluid layer for binding labels to the biological component such as a fluorescent label (either attached to the magnetizing particles or unbound thereto), a reagent fluid layer to prep a biological component for further analysis such as a master mix fluid layer to prep a biological component for PCR, and so on.

As used herein, a plurality of items, structural elements, compositional elements, and/or materials may be presented in a common list for convenience. However, these lists should be construed as though individual members of the list are individually identified as a separate and unique member. Thus, no individual member of such list should be construed as a de facto equivalent of any other member of the same list solely based on presentation in a common group without indications to the contrary.

Concentrations, amounts, and other numerical data may be expressed or presented herein in a range format. A range format is used merely for convenience and brevity and thus should be interpreted flexibly to include not only the numerical values explicitly recited as the limits of the range, but also to include individual numerical values or sub-ranges encompassed within that range as if numerical values and sub-ranges are explicitly recited. As an illustration, a numerical range of “about 1 wt % to about 5 wt %” should be interpreted to include not only the explicitly recited values of about 1 wt % to about 5 wt %, but also to include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting one numerical value. Furthermore, such an interpretation should apply regardless of the breadth of the range or the characteristics being described.

While the present technology has been described with reference to certain examples, various modifications, changes, omissions, and substitutions can be made without departing from the spirit of the disclosure. The disclosure is limited only by the scope of the following claims.

What is claimed is:

1. A fluid preparation device, comprising:
a fluid-receiving vessel; and
a fluid loader with multiple fluid chambers including a first fluid chamber and a second fluid chamber, the multiple fluid chambers partially defined by actuator seals that are positioned on an actuator, wherein the actuator is moveable among a plurality of positions and the actuator seals are positioned on the actuator to:
fluidically separate the first fluid chamber from the second fluid chamber when the actuator is in a closed position from the plurality of positions,
allow fluidic communication between the second fluid chamber and the fluid-receiving vessel when the actuator is in a first open position from the plurality of positions, and
allow fluidic communication between the first fluid chamber and the second fluid chamber when the actuator is in a second open position from the plurality of positions.

2. The fluid preparation device of claim 1, wherein the first open position and the second open position are the same position from the plurality of positions.

3. The fluid preparation device of claim 1, wherein the first fluid chamber is loaded with a first fluid and the second fluid chamber is loaded with a second fluid.

4. The fluid preparation device of claim 1, wherein the first fluid chamber is loaded with a first fluid having a first fluid density, the second fluid chamber is loaded with a second fluid having a second fluid density that has a greater density than the first fluid density, and the first fluid or the second fluid includes a particulate substrate dispersed therein, wherein when the second fluid and the first fluid are received by the fluid-receiving vessel, a multi-fluid density gradient column is formed in the fluid-receiving vessel.

5. The fluid preparation device of claim 1, wherein the first fluid includes a biological sample including a biological component that is associated with a surface of the particulate substrate.

6. The fluid preparation device of claim 1, wherein the particulate substrate includes magnetizing particles.

7. The fluid preparation device of claim 1, wherein fluidic communication from the first fluid chamber to the second fluid chamber is established via a first bypass channel allowing drainage from the first channel around a first actuator seal, and fluidic communication from the second fluid chamber to the fluid-receiving vessel is established via a second bypass channel allowing drainage from the second channel around a second actuator seal.

8. The fluid preparation device of claim 1, wherein the first fluid chamber, the second fluid chamber, and the fluid-receiving vessel include vents to permit air flow when the actuator is in the first open position or the second open position.

9. The fluid preparation device of claim 1, further comprising a magnet that is spatially located adjacent to the fluid-receiving vessel to provide a magnetic field across the fluid-receiving vessel.

10. The fluid preparation device of claim 1, wherein the fluid-receiving vessel includes a capillary force gradient portion.

11. The fluid preparation device of claim 10, wherein the capillary is loaded with a fluid and is layered adjacent to a second fluid along a capillary force gradient portion.

12. A particulate substrate fluid density layering system, comprising:
a first fluid having a first fluid density;
a second fluid having a second fluid density that is denser than the first fluid density;
a particulate substrate dispersed or dispersible in the first fluid or the second fluid; and
a fluid loader with multiple fluid chambers partially defined by actuator seals that are positioned on a common actuator, wherein when the actuator is in a closed position, a first fluid chamber containing the first fluid and a second fluid chamber containing the second fluid are isolated from one another, and when the actuator is in an open position, one or both of the first fluid or the second fluid is allowed to drain from the first fluid chamber or the second fluid chamber, respectively.

13. The particulate substrate fluid density layering system of claim 12, wherein the particulate substrate includes magnetizing particles that are dispersed or dispersible in the first fluid or the second fluid, wherein the magnetizing particles include a surface to associate with a biological component of a biological sample or are surface to a biological component of a biological sample.

14. The particulate substrate fluid density layering system of claim 13, wherein the first fluid chamber is pre-loaded with the first fluid which includes magnetizing particles dispersed therein, or wherein the first fluid chamber is pre-loaded with dry magnetizing particles and the first fluid is loadable to the first fluid chamber so that the first fluid includes the magnetizing particles dispersed therein.

15. The particulate substrate fluid density layering system of 12, further comprising a fluid-receiving vessel integrated with the fluid to receive the multi-fluid density gradient column above a pre-loaded elution buffer or a master mix for nucleic acid process positioned.

16. A method of forming a multi-fluid density column, comprising:
loading a first fluid chamber of a fluid loader with a first fluid having a first fluid density, wherein the first fluid chamber is separated from a second fluid chamber when an actuator of the fluid loader is in a closed position, wherein the second fluid chamber contains a second fluid or the second fluid is loadable to the second fluid chamber via a port, wherein the second fluid has a second fluid density that is denser than the first fluid density, and wherein the second fluid chamber is separated from other fluid chambers when the actuator of the fluid loader is in the closed; and
moving the actuator from the closed position to one or more sequential open positions to drain the second fluid followed by the first fluid in series to form a multi-fluid density column with first fluid remaining separated above the second fluid along a density-differential fluid interface.

17. The method of claim 16, further comprising combining the first fluid with a particulate substrate either in the first fluid chamber or prior to loading the first fluid to the first fluid chamber, wherein the particulate substrate includes a surface to become associated with a biological component of a biological sample or where the surface is associated with the biological component of the biological sample.

18. The method of claim 16, wherein the multi-fluid density gradient column is formed in a fluid-receiving vessel positioned adjacent to a magnet, wherein the magnet is actuatable to move magnetizing particles included in the first fluid or the second fluid along the multi-fluid density gradient column.
